(19) United States
(12) Patent Application Publication
(10) Pub. No.: US 2010/0204791 A1
(43) Pub. Date: Aug. 12, 2010

(54) SYSTEM FOR LIFTING AND RESHAPING SOFT TISSUE

(75) Inventors: Adi Shfaram, Herzliya Pituach (IL); Eyal Gur, Ganei Tikva (IL); Adi Cohen, Hod Hasharon (IL)

Correspondence Address: THE NATH LAW GROUP, 112 South West Street, Alexandria, VA, 22314, US

(73) Assignee: ORBIX MEDICAL LTD., Kiryat Shmona (IL)

(21) Appl. No.: 12/704,011

(22) Filed: Feb. 11, 2010

Related U.S. Application Data
(63) Continuation-in-part of application No. 10/516,685, filed on Dec. 6, 2004, now Pat. No. 7,670,372.
Application No. 10/516,685 is a continuation-in-part of application No. PCT/IL2008/001360, filed on Oct. 22, 2008.

(30) Foreign Application Priority Data
Jun. 11, 2002 (IL) 150151

(51) Int. Cl. A61F 2/12

(52) U.S. Cl. 623/8

(57) ABSTRACT

A system and method for shaping/reshaping and lifting a soft tissue, comprising a cradling member configured to cradle the soft tissue, an anchoring system comprising one or more anchors for fixing the cradling member to a posture tissue, and adjustable suspension members suspended from the one or more anchors.

This is a Continuation-In-Part of U.S. application Ser. No. 10/516,685, filed Dec. 6, 2004 as National Phase of PCT/IL2003/000490 filed Jun. 11, 2003 and which claims priority from Israeli Patent Application no. 150151 filed Jun. 11, 2002, and of International PCT Application No. PCT/IL2008/001360 filed Oct. 22, 2008 which claims priority from U.S. Provisional No. 60/960,831 filed Oct. 16, 2007, the contents of which are hereby incorporated by reference in their entirety.

### FIELD OF THE INVENTION

The present invention is generally in the field of soft tissue and organ support, lift and shape correction.

This invention further relates to the field of breast shape correction and more specifically it is concerned with breast lifting and supporting.

The system according to the present invention may be applied for the purpose of shape correction, lifting and supporting of breast or other soft tissues such as buttocks, arm or neck tissues, by altering the shape of an individual's breast or other soft tissues such as buttocks, arm or neck tissues.

### BACKGROUND OF THE INVENTION

Over the years, factors such as pregnancy, nursing, and the force of gravity take their toll on a woman's breasts. This situation is known as ptosis and is defined as a situation at which the nipple-areola complex projection is lower than the infra-mammary fold, i.e. the nipple is below the level of the lower breast crease.

As the skin loses its elasticity, the breasts often lose their shape and firmness and begin to sag, obtaining a teardrop like shape rather then a cone-like shape. Breast lifting, also referred to by the alternative names mastopexy and mammoplasty, is a procedure used for reshaping saggy and loose breasts, elevating the nipple and areola to a higher level and, thus affording the breast its former shape and firmness which can result in a revitalized body image that can bolster a woman's self-esteem.

Many women use a push-up bra to support their breasts. This however is at times uncomfortable and may be restricting as far as the selection of clothing.

Mastopexy procedure according to prior art techniques involves reducing ptosis (sagging of the breast caused by stretched skin, in many cases due to a great loss of breast tissue). During a common procedure for breast lift, long incisions are made along the natural creases in the breast and around the dark skin surrounding the nipple (areola), a keyhole-shaped incision above the areola is also made to define the new location for the nipple. Excess skin is removed from the lower section of the breast and the areola, nipple, and underlying breast tissue are repositioned up to a higher position. The nipple is moved and incisions are closed with sutures.

Several methods for performing a mastopexy are known, and the technique opted for depends mainly on the amount of breast and fat tissue, the amount of skin to be removed, symmetry in volume of breasts and size of areolas, and choice and taste of patient. Since the procedure involves surgery, it may be coupled with breast augmentation and with resizing or repositioning of the areola to a more aesthetically pleasing position and the shape of the mound may be improved by placement of breast implants. Occasionally, one-side of the breast requires higher lifting than the other breast, to correct asymmetry.

U.S. Pat. No. 5,676,161 to Breiner discloses a mastopexy procedure according to which an anchor-shaped incision is made, having a bottom line along the infra-mammary crease, using a circular cutter to form the top portion of the incision and an incision around the areola to reduce the diameter thereof. After removing excess skin inside the incision, and breast tissue in the case of mammaplasty, shifting the areola, nipple and underlying breast tissue upward to position the areola/nipple complex within the circular top portion, pulling the flaps of skin formed to the sides of the incision down and around the areola and underneath same, and then suturing adjacent skin edges to complete the lifting and reshaping.

U.S. Pat. No. 5,584,884 to Pignataro discloses a mammary prosthesis comprising a wedge shaped sheet of flexible biocompatible material having reinforced upper and lower attachment portions for attachment to bone of a patient by bone anchors, with the lower attachment portion being anchored to one or more ribs. The lower attachment portion includes a support member less flexible than the sheet material having suture receiving openings for receiving bone anchor sutures.

U.S. Pat. No. 5,217,494 to Coggins et. al. discloses a prosthesis for supporting tissue which among others may be used also in a breast lift procedure, wherein one end of the prosthesis is implanted deep into the breast tissue and the opposed end is attached to either the clavicle or the rib.

These procedures are typically performed under general anesthesia, though at times local anesthesia is sufficient, and they may last several hours, depending on the extent of the surgery.

As with any surgery, there is always a possibility of complications such as a reaction to the anesthesia, bleeding and infection (which may cause scars to widen). Mastopexy does leave noticeable, permanent scars, although are so planed as to be concealed by a woman's bra or bathing suit. One may expect that after about one year the scars will hardly be noticeable. As far as aesthetics, there may also be some unsatisfactory, as the final appearance may not always meet the patient's expectations. Evermore, a breast lift performed according to conventional techniques won't keep firm forever, the effects of gravity, pregnancy, aging, and weight fluctuations will eventually take their toll again.

Other types of soft tissues and organs affected by forces of gravity, aging and obesity are also surgically treated to achieve a healthier and aesthetic appearance.

An arm lift, also known as brachioplasty, is a surgical procedure to remove loose skin and excess fat deposits along the upper arm. Brachioplasty is performed to remove excess tissue and reduce the circumference of the upper arm. Such surgical procedure bears a possibility of complications such as a reaction to the anesthesia, bleeding and infection (which may cause scars to widen).

A neck lift, or platysmaplasty and even submental platysmaplasty, is a surgery designed to reduce the loose look of sagging skin, or a “turkey waddle”, in the neck area and under the jaw line. In such a procedure sections of skin are trimmed and lifted into place and sutured or fixed with biological tissue glue. With the platysmaplasty, a section of fat and muscle is removed and the ends are sutured to bring them together at the mid-anterior (front) section of the neck. The skin can be brought together under or behind the ear to further firm up appearance of the neck. According to some surgical procedures, suture, mesh or even AlloDerm suspension as a sort of hammock are used to keep the neck tight and waddle-free. Brachioplasty and platysmaplasty involved massive surgical procedure with the related risks and often leave noticeable, permanent scars and often some unsatisfactory results, as the final appearance may not always meet the patient's expectations

### SUMMARY OF THE INVENTION

The presently disclosed subject matter relates to systems and methods for lifting, shaping, supporting and/or reshaping of soft tissues.

In accordance with one aspect of the present invention there is provided a system and a method for breast lifting, reshaping and supporting. The system according to the invention and the method for carrying it out may be minimal invasive and may be considered as non-surgical, i.e. they do not involve incisions (but rather two or four stab incisions) and removal of excess skin, nor do they require stitches. However, the system may also be used in conjunction with breast enlargement (breast augmentation with mastopexy), breast reshaping or breast size reduction or for correction of pseudo ptosis, which are typically invasive surgical procedures.

The system and method in accordance with the invention may be utilized for organs/areas of the human body such as arms, neck, buttocks and other areas with soft tissues affected by factors such as aging, weight lose, body changes, gravity force or obesity, which take their toll on these tissues.

One aspect of the present invention provides a system for breast lifting, reshaping and supporting wherein one or more anchors are fixed to a posture tissue, above the desired nipple level with one or more suspending members cradling the breast and extending from the one or more anchors, such that tensioning the suspending members entails lifting and supporting of the breast.

The posture tissue is a bone (ribs or collar bone), though it may also be a muscle tissue (pectoral or intercostals or fascia). For each breast, anchors may be fixed to one or more posture, depending on the physiology/anatomy of the patient, the shape of the breast prior to lifting, and the desired lifting result. An anchor may be a bolt fixture or a threaded fixture typically but not restricted thereto, a self tapping screw) for screw-fixation into a bone, a suspending hook for bearing from a bone, i.e. clinging from the bone, or a clasp formed with hooks for grasping soft tissue (muscle or fascia). Alternatively, where the posture tissue is a muscle or fascia, the suspension member may be fixed thereto by stitching. According to still an alternative, the suspension member may be attached to a bone by tying or yarning it through a bore formed through the bone.

A suspension member according to the invention may be a tendon-like wire or a mash, made of organic material (e.g. tendons), or synthetic material (e.g. silicone, Gortex™, etc.) or a combination thereof.

According to an embodiment of the invention, the suspension member is formed with or comprises a cradling portion or a cradling member, respectively, having increased surface area than the suspension member, for supporting the breast or organ/soft tissue from below and padding it, said cradle being a mesh, a strap or a tube-like member mounted on a wire-like suspension member.

A significant advantage of the present invention, apart from the fact that in some cases it can be a so-called minimal invasive procedure, is that the aesthetic results and longevity results and appearance may be modified to match with customer's expectations during, or any time after the procedure, i.e. corrections may be easily effected after a while (typically several years), if ptosis reoccurs.

The proposed procedure approves well with other medical procedures and may be combined with breast enlargement (augmentation) or breast reduction. Even more so, the procedure is completely reversible. Apart from that the procedure is fast and relatively inexpensive, and the patient is dismissed shortly after, leaving practically no scars in the so-called minimally invasive procedure.

The procedure according to an aspect of the present invention is carried out by inserting a tool through one or two locations at a bottom face of the breast or organ/body part, directed towards the posture tissue such that it passes through the breast tissue organ/body part. The tool is used for fixing the anchor to the posture, and according to an embodiment thereof, the tool can also be used for guiding and manipulating the suspension member so as to cradle the breast or organ/body part and then tensioning the suspension member at the required extent.

In some cases, depending among others on breast or organ/body part size, two sets of such system may be required for a breast or organ/body part, so as to improve the under support.

According to a further aspect of the present invention there is provided a method for breast lift or organ/body part, said method comprises the following steps:

a) introducing through the breast or organ/body part at least one anchor and fixing it to a posture tissue;

b) yarning a suspension member through the breast or organ/body part; said suspension member extending from said at least one anchor and passing within tissue at a bottom portion of the breast or organ/body part so as to cradle the breast or organ/body part;

c) tensioning the suspension member to thereby lift the breast or organ/body part; and

d) fixating the length of the suspension member.

An alternative method comprises the following steps:

a) yarning a cradling member of a suspension member through the breast or organ/body part, at a bottom thereof, such that loose ends thereof extend from the breast or organ/body part;

b) fixing at least one anchor to a posture tissue, at a level above the desired nipple level or center of gravity of the organ/body part;

c) attaching a suspension member to the at least one anchor;

d) articulating the loose ends of the cradling member to the suspension member and fixedly adjusting the length thereof.

According to an embodiment, two anchors are fixed to the posture tissue, with a suspension member extending from each one, such that the loose ends of the cradling member are articulated to each respective end of a suspension member. Still preferably, excessive ends of the suspension members and of the cradling member, are trimmed.

The method according to an embodiment thereof further comprises fitting a cradle member on the suspension member, for supporting the breast from below. According to one application, the cradle member is a strap-like portion (possibly a mesh-like material) continuously extending from the suspension member. According to a different application, the cradle member is integral with or mounted over a supplementary member fixedly attached to the suspension member, whereby the breast height is set by adjusting the relative length of the suspension member and the supplementary member.

Where the system is used in conjunction with a breast or other soft tissue or organ enlarging procedure, the cradling member may directly support the implant.

Using the system in accordance with the present invention in a minimally invasive procedure in accordance with the present invention has several possible advantages, namely:

a) the mere incisions are two or four stab incision, which are fast healing and practically leave no scars;

b) the procedure is considerably shorter than any prior art procedure;

c) the procedure may be carried out under local anesthesia, whereby the patient may be dismissed shortly after;

Moreover, the system in accordance with the invention may also be used when performing a non minimally invasive procedure, and may present the following results:

d) the procedure renders the soft tissue with a longer and better aesthetic effect;

d) the procedure is adjustable and reversible at any time.

For carrying out the procedure of the present invention, there is provided a tool kit which depending on the specific procedure will include one or more of the following tools:

a) tool for stabbing the breast and yarning a cradling member therethrough, such that its free ends extend from the breast; the same tool may be used for creating a passage through the breast for the suspension member;

b) tool for deploying and fixing the anchors to a posture tissue (bolting or screwing in the case of a bone posture; stitching in the case of muscle tissue); this toll is also used for yarning the suspension member which is articulated to the anchor;

c) tool for articulating and tensioning ends of the cradling member to the suspension members, and for adjusting the length, fixating and trimming excessive ends.

However, according to different applications, rather than using a tool, slack end of the cradling member and the corresponding slack end of the suspension member, may be manually tensioned and tied to one another, their ends being trimmed by conventional means.

The present invention further calls for a tool useful in carrying out a procedure according to the invention, said tool adapted for tensioning and clamping cord-like slack ends of the cradling member and a corresponding suspension member; said tool comprising a housing fitted at a fore end with a cord receiving opening for receiving two or more cords, a clamp deploying mechanism for clampingly articulating the at least two cords, and a cord trimming mechanism for trimming the cords adjacent the clamp.

According to another aspect of the present invention there is provided a kit for performing a breast-lift procedure, the kit comprising at least one set of suspension members, anchoring means for anchoring a suspension member to a posture tissue, and means for tensioning and fixating the suspension member. The kit may also comprise one or more breast-cradling/padding members and one or more tools, e.g. a tool to facilitate tensioning clamping and trimming of a suspension member and a cradling member.

According to yet another aspect of the present invention, there is provided a system and a method for lifting and shaping/reshaping soft tissue such as buttocks tissues, arms and neck.

The term soft tissue as used herein, refers to any tissue of the organ and surrounding the organ to be lifted. For example the tissue may comprise muscle, fat, fibrous tissue, blood vessels, or other supporting tissue of the body.

The system according to the invention and the method for carrying it out may be minimally invasive and may be considered as non-surgical, i.e. they do not involve extensive incisions and removal of excess skin, nor do they require stitches (but rather the process involves several stab incisions). However, the system may also be used in conjunction with typical soft tissue enlargement/reduction, lifting and reshaping surgeries typically performed in a so-called open procedure.

A soft tissue shaping/reshaping system according to this aspect of the present invention comprises a cradling member for cradling the soft tissue, and an anchoring system for fixing the cradling member to posture tissue in at least one location above the center of gravity of the soft tissue.

The anchoring system in accordance with an aspect of the invention comprises one or more anchors configured to be fixed to a posture tissue and at least one support member provided on the cradling member. The at least one support member in accordance with an embodiment of the invention is configured for barring against, and fixing to, body tissue adjacent the lifted tissue.

In accordance with yet another aspect of the present invention, there is provided a system for reshaping, supporting and lifting a soft tissue. The system in accordance with this aspect comprises one or more anchors adapted to be fixed to a posture tissue located above a center of gravity of the soft tissue; two or more readily adjustable suspension members suspended from the one or more anchors, each readily adjustable suspension member having a free end; and a cradling member having a first end and a second end and being configured to cradle the soft tissue, the first end and the second end of the cradling member being integral with and/or movably attached to the free end of a respective suspension member, and the cradling member having a width greater than the width of each of the two or more readily adjustable suspension members. The system being configured to lift, support and impart the soft tissue with a desired shape.

In accordance with yet an aspect of the invention, there is provided a system for reshaping and lifting a soft tissue. The system in accordance with this aspect comprises one or more anchors adapted to be fixed to a posture tissue above a center of gravity of the soft tissue; two or more readily adjustable suspension members having an anchoring end, and a cradling end, the two or more readily adjustable suspension members being suspended from the one or more anchors at the anchoring end and being movably attached or fixed to the one or more anchors. The system further comprises a cradling member having a first end movably attached to and/or integral with the cradling end of one of the two or more readily adjustable suspension members, and a second end movably attached to and/or integral with the cradling end of one of the two or more readily adjustable suspension members. The cradling member in accordance with this aspect is configured to cradle the soft tissue and has a width greater than the width of each of the two or more readily adjustable suspension members; wherein the system is configured to lift and impart the soft tissue with a desired shape.

Any one or more of the following features and characteristics may be implemented in the systems and method according to the present invention:
the soft tissue may be a breast tissue, arm tissue, neck tissue or buttocks tissue or other soft tissue;
the soft tissue may be a breast tissue and the posture tissue is located at or above a desired nipple level;
the posture tissue is located at or above the center of gravity of the soft tissue;
the soft tissue may be buttocks tissue and the anchoring system may comprise one or more anchors adapted to be fixed to a posture tissue, with one or more suspending members suspended from the one or more anchors and extending through a tissue for supporting the cradling member;
the anchor may be a bolt fixture fixed to a posture tissue, in case of breast lifting the posture tissue may be a rib, a collar bone or fascia, in case of the reshaping of the buttocks tissue the posture tissue may be a Pelvic girdle bone, in case of the reshaping of the neck tissue the posture tissue may be a mandibular bone, or in case of the reshaping of the arm tissue the posture tissue may be a humerus bone.
the soft tissue may be breast tissue and the cradling member may be attached to a posture tissue along an infra-mammary fold using the support members;
the soft tissue may be breast tissue and the cradling member may be attached to a posture tissue along an infra-mammary fold and may further comprise an anchoring system positioned at or above the desired nipple level with one or more suspending members suspended from the one or more anchors wherein the anchor may be a bolt fixture fixed to a rib or a collar bone;
the one or more support members are integral with or articulated to the cradling member;
the support members may be in the form of tabs laterally projecting from a longitudinal edge of the cradling member fitted for bearing against posture tissue;
at least a portion of the cradling member may be made of a bio-compatible material, mesh like material, silicon sheet, silicon sheet comprising embedded mesh-like material, reinforced silicon material, silicon sheet with mesh-like material with non-homogeneous reinforcing qualities etc;
the cradling member comprises at least one reinforced zone;
the first and second ends of the cradling member are reinforced;
the material from which the cradling member is formed is a silicon material and the reinforced zone is achieved by providing the material with at least one reinforcing member in the form of a mesh-like material;
the cradling member is homogeneously/non-homogeneously reinforced;
the one or more suspending members are attached to the cradling member such that the load exerted on a suspending member-cradling member connecting point is spread over a portion of the cradling member; and
the one or more suspending members is stitched into the cradling member using more than one stitch.

### BRIEF DESCRIPTION OF THE DRAWINGS

In order to understand the invention and to see how it may be carried out in practice, some embodiments will now be described, by way of non-limiting examples only, with reference to the accompanying drawings, in which:

FIG. 1 is a partially sectioned side view illustrating the anatomy of an erect, firm breast;

FIG. 2 is a side view illustrating a saggy breast;

FIGS. 3A to 3H are consecutive steps of carrying out a breast lift procedure according to a first example of the present invention;

FIGS. 4A to 4G are consecutive steps of carrying out a breast lift procedure according to a second example of the present invention;

FIG. 5A is a side section through a saggy breast prior to breast lift;

FIG. 5B is a side section through the breast in FIG. 5A, after breast lift;

FIG. 6 is a front view of a female body, superimposing how the breasts are supported by two suspension members;

FIGS. 7A to 7C are schematic side views illustrating different examples of anchoring means;

FIGS. 8A to 8D illustrate a surgical tool useful in carrying out a breast-lifting procedure, in four consecutive operative positions;

FIG. 9A schematically illustrates how the present invention is used for reshaping an individual's breast tissue according to an example of the present invention;

FIG. 9B illustrates is a plan view of the cradling member according to an example of the present invention;

FIG. 9C illustrates a plan view of the cradling member according to yet an example of the present invention;

FIG. 9D illustrates a portion of the cradling member fitted with a suspension member according to one example of the present invention;

FIGS. 10A and 10B schematically illustrate how the ends of the cradling member are fitted with suspension members during the procedure according to one example of the present invention;

FIG. 11A-11B schematically illustrate how the present invention is used for reshaping an individual's buttock tissue according to one example of the present invention illustrating a cradling member according to one example of the present invention;

FIG. 12 schematically illustrates how the present invention is used for reshaping an individual's arm tissue according to an example of the present invention; and

FIG. 13 schematically illustrates how the present invention is used for reshaping an individual's neck tissue according to an example of the present invention.

### DETAILED DESCRIPTION OF EMBODIMENTS

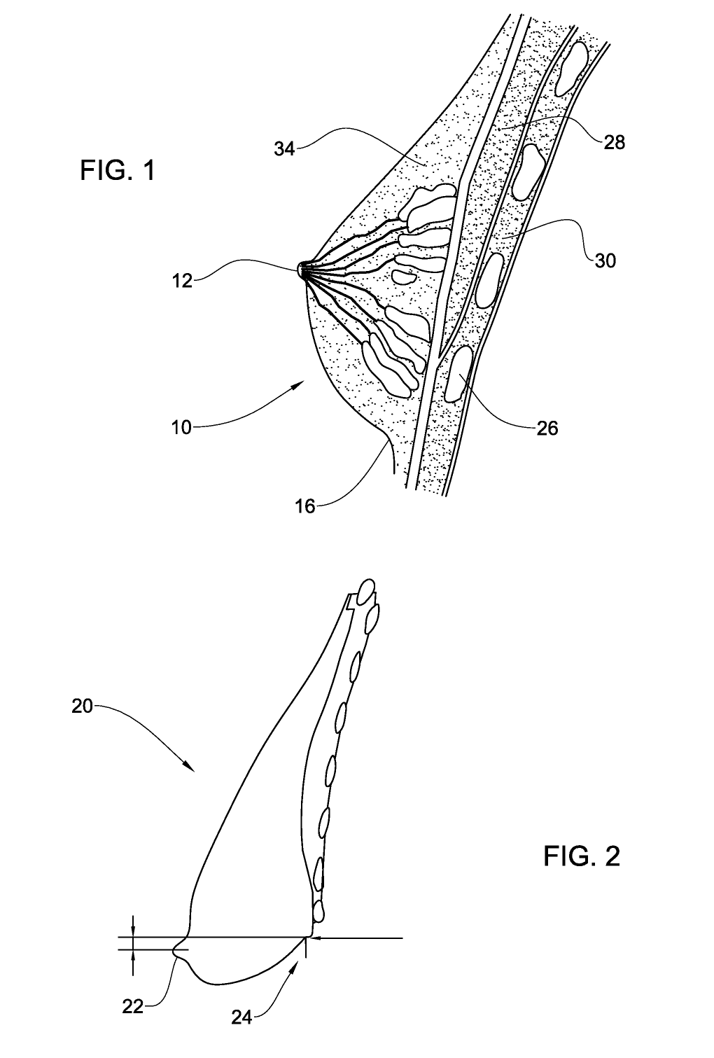

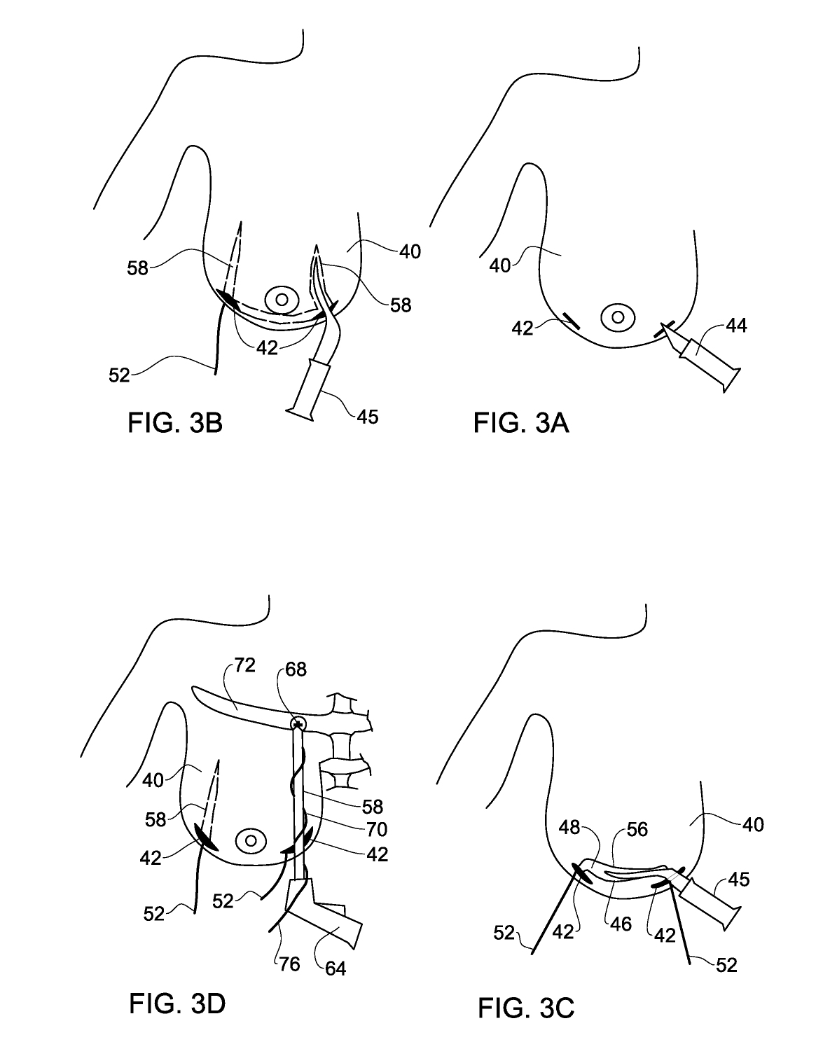

The breast 10 seen in FIG. 1 is firm and erect though of small size. One can notice that the nipple 12 is facing forward and that the nipple-areola complex projection is higher then the infra-mammary fold 16, i.e. the nipple 12 is well above the level of the lower breast crease 16. Contrary thereto, the breast 20 of FIG. 2 is saggy and it is noticed that the nipple 22 is facing downward and extends below the infra-mammary fold 24.

Reverting to FIG. 1, one can further notice several ribs 26, pectoral muscle 28, intercostals muscles 30 and the fat tissue 34.

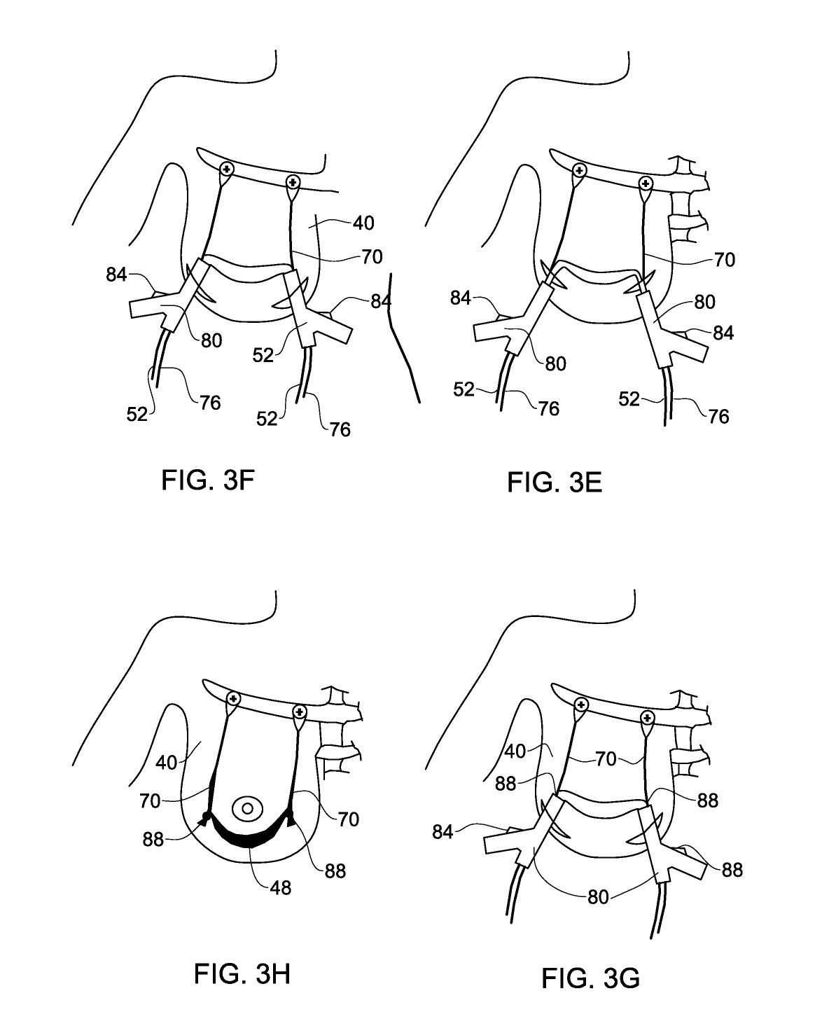

Turning now to FIGS. 3A to 3H, there is illustrated a sequence of carrying out a breast lift according to a first embodiment of the present invention.

At a first step, after local anesthesia of the beast 40, two stab incisions 42 are formed at a bottom portion of the breast, with a surgical tool 44 (FIG. 3A). Then using another tool 45, a passage 46 is formed between the incisions and using that tool a cradling member 48 is yarned through the passage 46, such that its slack ends 52 extend through the incisions 42 (FIG. 3B). As seen in FIG. 3B, the cradling member 48 comprises a central band-like portion 56 adapted for cradling and supporting the breast from below, and two yarns 52 extending at each side thereof. The band-like portion 56 as well as the yarns 52 may be made of organic material, e.g. tendons, or synthetic material e.g. Gortex™, etc.

Using tool 45, two passages 58 are made (FIG. 3C), preferably extending from the incisions 42. It is however noticed that the passages 58 may be formed before inserting the cradling member 48. Through the passages 58 an anchoring tool 64 is applied (FIG. 3D), said tool 64 being pre-fitted with an anchoring screw 68 (preferably a self tapping screw) and a suspension member in the form of a cord 70 attached to the anchor screw 68. By means of tool 64 the anchor screw 68 is screwed to rib 72. The same procedure takes place through both passages 58, with slack ends 76 of the suspension members extending from the incisions 42.

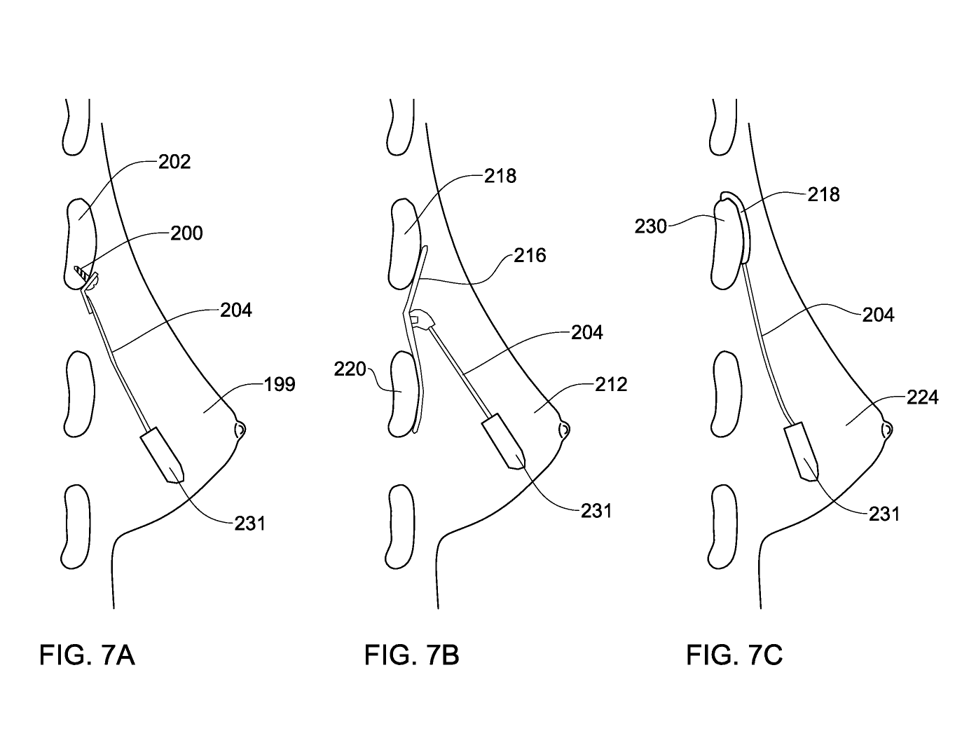

Using a different tool designated 80 (such a tool is disclosed in more detail with reference to FIGS. 7A to 7D), slack end 52 of the cradling member and the corresponding slack end 76 of the suspension member, are articulated, simultaneously at both sides of the breast (FIG. 3E). Preferably while the patient is in an upright position, tensioning begins using the tool 80, by repeatedly pulling trigger 84, until the breast elevates to a desired position. This action provides real time indication of the breast's new form and position, and even more so, the patient may take part in deciding to what extent to lift the breasts.

Once the breast 40 assumes the desired repositioning and form, a fixing clamp 88 is applied by tool 80 (FIG. 3G), and by further manipulating trigger 84 of the tool 80, the slack ends 52 of the cradling member and the corresponding slack ends 76 of the suspension member are trimmed (FIG. 3H).

The tool is then removed and shortly after the patient may be dismissed, with complete healing expected in a matter of days, essentially not leaving any noticeable scars.

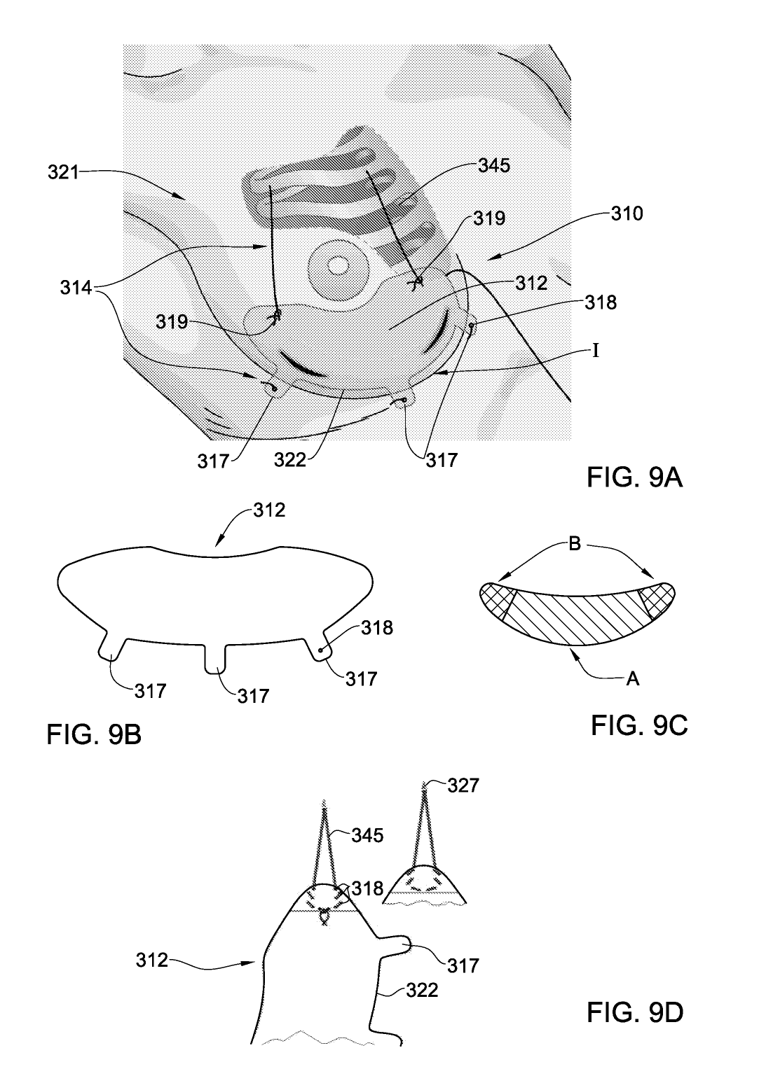

According to a different application, rather than using a tool, the slack end 52 of the cradling member and the corresponding slack end 76 of the suspension member may be manually tensioned and tied to one another and the cradling member (e.g. as seen in FIGS. 10A and 10B), their ends being trimmed by conventional means, as known in the art of suturing.

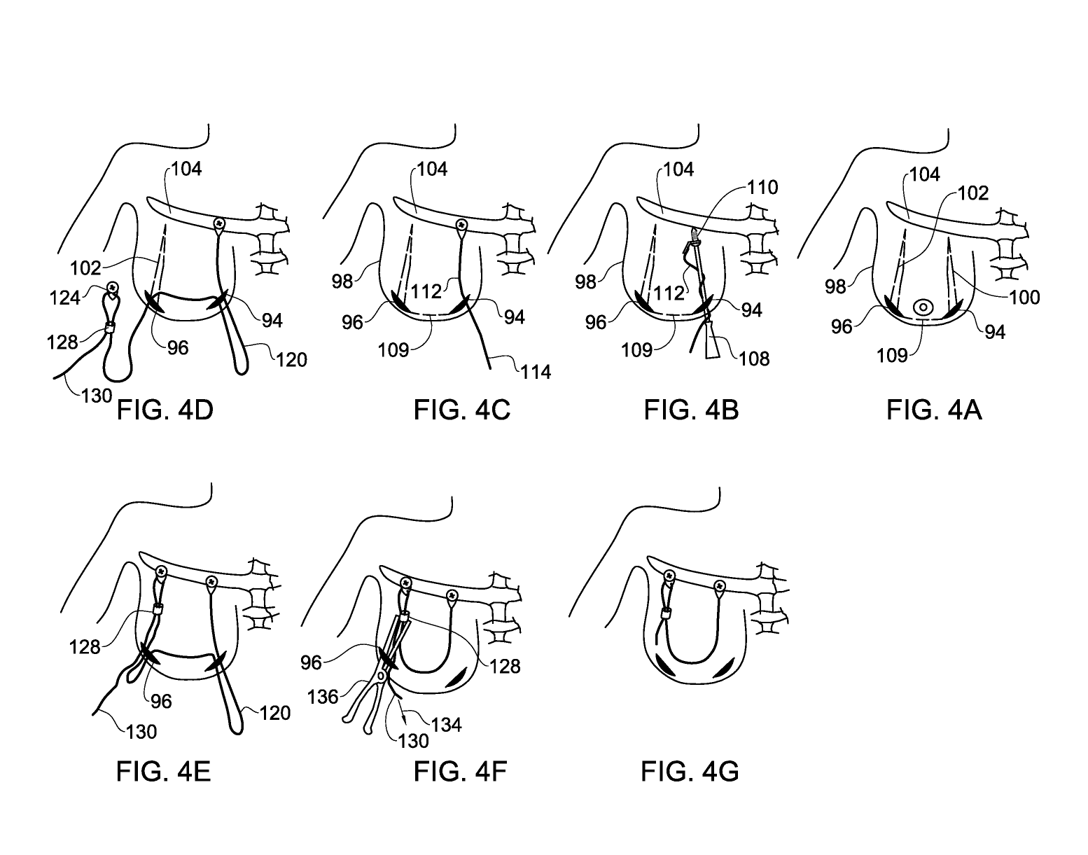

Further attention is now directed to FIGS. 4A to 4G, illustrating still another method for carrying out a breast lift according to the present invention.

First, two stab incisions 94 and 96 are made at a bottom portion of the breast 98, and a corresponding passage 100 and 102 is formed extending towards a suitable posture tissue, rib 104 in the present example (FIG. 4A). A transverse passage 109 is formed between the incisions 94 and 96. Then a tool 108, resembling a screwdriver, is introduced through the first passage 100, said tool carrying at its fore end an anchor in the form of screw 110, to which a suspension cord 112 is attached. The anchor is fixed to the rib 104 (FIG. 4B), and the tool 108 is removed leaving a long free end 114 of the suspension member, extending from the incision 94 (FIG. 4C). Preferably, tool 108 is fitted with a screw holding member (not shown), to prevent the screw 110 from departing from the tip of the tool.

The free end 114 is then yarned back through the incision 94, through the transverse passage 109 and out through the second incision 96, leaving a looped portion 120 extending from the first incision 94 (FIG. 4D). A second anchor 124 is then provided, which comprises an eye for slidingly receiving the suspension cord 112, and a clamp 128 is loosely mounted over two overlapping portions of the suspension cord (FIG. 4D). Using the same tool 108 (not shown in FIG. 4D), the second anchor 124 is introduced through the second passage 102 and is then attached to the rib 104, whilst the loose end 130 of the suspension cord extends from incision 96 (FIG. 4E).

The loose end 130 is then pulled in direction of arrow 134 (FIG. 4F), shrinking the looped portion 120 and tensioning the suspension cord until the breast is deformed and lifted to a desired extent. At this point, a clamping tool 136 is introduced through incision 96, to shrink the clamp 128, so as to thereby fixate the tension of the suspension cord.

As mentioned in connection with the embodiment of FIG. 3, the tensioning step (FIG. 4F) is preferably carried out while the patient is in an upright position, such that actual indication is available regarding the breast's new form and position, and even more so, the patient may take part in deciding to what extent to lift the breasts.

The loose end 130 is then trimmed, possibly by tool 136 (FIG. 4G) and the procedure is complete. Shortly after the patient may be dismissed, with complete healing expected in a matter of days, essentially not leaving any noticeable scars.

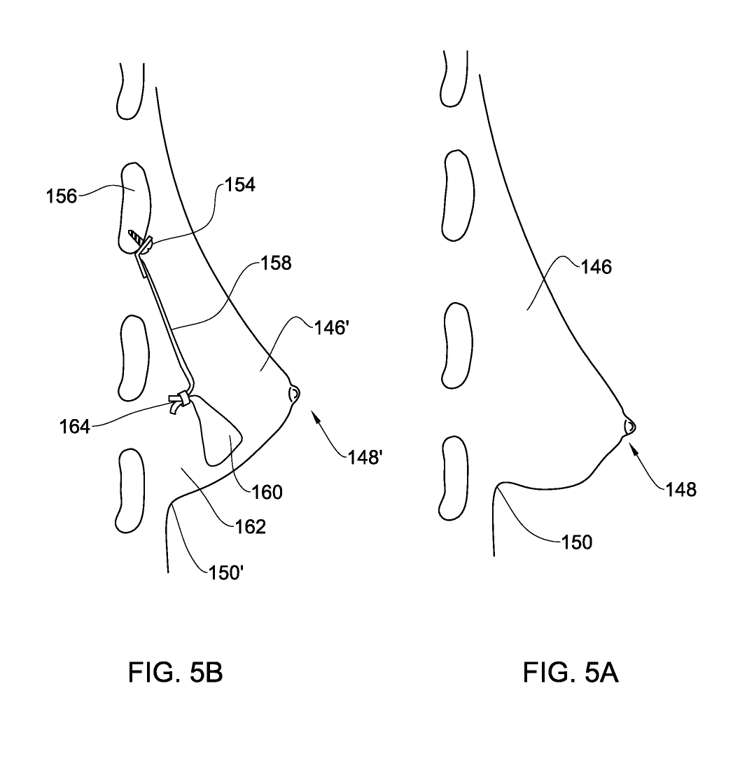

Turning now to FIGS. 5A and 5B, there is illustrated a breast 146 which in FIG. 5A is prior to beast lift and it is noticeable that nipple-areola complex 148 projection is lower than the infra-mammary fold 150. However, in FIG. 5B the same breast, now designated 146′ has undergone a breast lift procedure in accordance with the present invention and in this Figure the nipple-areola complex 148′ projection is higher than the infra-mammary fold 150′. Further noted in FIG. 5A, a screw type anchor 154 is screw fixed into a rib 156, with a suspension member in the form of cord 158 being attached thereto. A cradling member 160 extends through the fat tissue 162 of the breast, supports the breast from below, said cradling member being tensioned and articulated to the suspension member by clamp 164, thus lifting the breast and imparting it a firm and erect appearance.

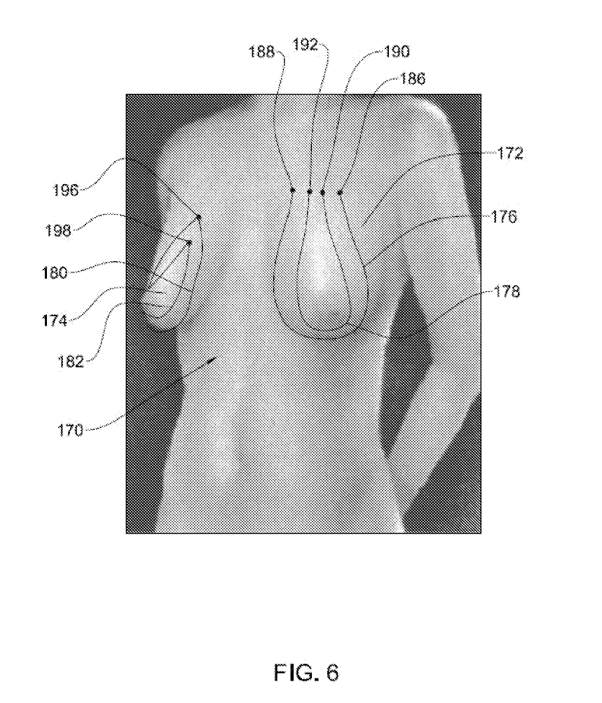

In FIG. 6, a female's thorax 170 is seen in which both the left and right breast 172 and 174 respectively, are supported by two sets of breast-lifting systems (176 and 178 for the right breast; and 180 and 182 for the left breast), each breast-lifting system being substantially similar to the systems disclosed herein above. The difference resides in that each breast is supported by two sets and it is noticed that whilst in the left breast 172 each suspension member is anchored by two distinct anchors (186; 188 for breast-lifting system 176, and 190; 192 for breast-lifting system 178), in the right breast 174 each suspension member is anchored by only one, common anchor (196 for breast-lifting system 180, and 198 for breast-lifting system 182).

FIGS. 7A to 7C illustrate 3 breasts, each lifted by a breast lifting system according to the invention, each fitted with a different anchoring means for attaching the suspension member to a posture tissue. In breast 199 of FIG. 7A the anchor is a threaded anchor 200 screw-coupled into a rib 202, with a suspension member 204 attached to the anchor. It is appreciated that the screw-type anchor may be a self-screwing thread type, i.e. does not require preparatory boring in the bone tissue, and there may be a bolt-type anchor, i.e. of the type which is inserted into a bore pre-drilled in the rib.

The breast 212 in FIG. 7B is supported by a system in which a brace-type anchor 216, which is attached to two neighboring ribs 218 and 220, e.g. by screws, etc. in FIG. 7C the breast 224, is supported by a system according to the invention in which the anchor is a suspending hook 228 bearing (clinging) from rib 230. However, as mentioned hereinabove, the anchoring member may also be attached to muscles e.g. by stitching or by a clasp, etc.

The cradling member 231 illustrated in the figures may be, for example, a tube-like member mounted on the suspension member 204, for increasing the sectional area thereof.

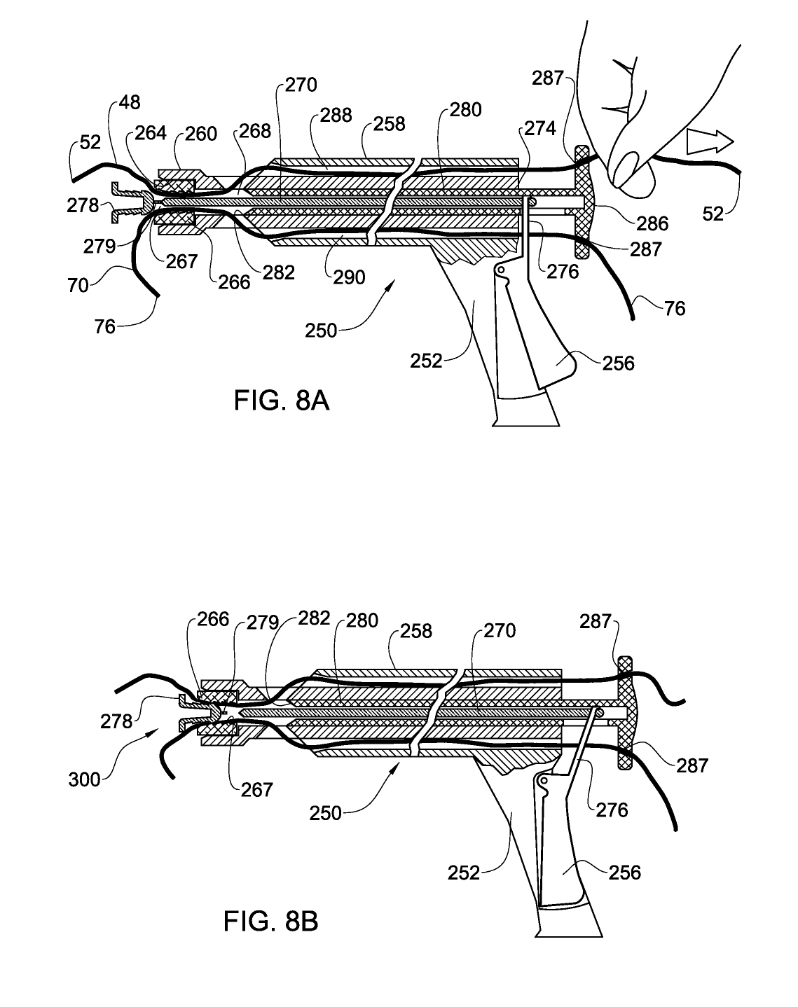

According to another aspect of the invention, there is provided a surgical tool useful in carrying out the method disclosed with reference to FIGS. 3A to 3H. A tool 250 is illustrated in more detail with further reference to FIGS. 8A to 8D.

The tool 250 has a general pistol-like shape having a gripping portion 252 fitted with a clamping trigger 256, and a stein portion 258 having a fore end 260. At least the fore end is sized for inserting through a stab-incision formed in a breast, as illustrated for example in FIGS. 3E to 3G The fore end 260 is fitted with a clamping unit receptacle 264 (best seen in FIG. 8D), accommodating an annular locking member 266 having a serrated inner bore 267 sized to receive therethrough an end of the cradling member 52 and a corresponding end of the suspension member 76 (see FIG. 3E). The stem portion 258 comprises a central bore 268 accommodating an elongate plug-stem 270 extending from the clamping unit receptacle 264 towards a rear end 272 of the stem portion, where it projects and is articulated to a lever 276 of the trigger 256. A plug 278 is integrally connected by a tear zone 279 to a fore end of the plug-stem 270. The plug 278 is serrated and sized for snapingly locking within the locking member 266.

Coaxially received within the central bore 268 there is a trimmer 280 having a trimming end 282 at a fore end and a pusher 286 at its rear end, said pusher comprising two bores 287. Two bores 288 and 290 extend through a portion of the stem portion 258, fitted for receiving the end of the cradling member 52 and a corresponding end of the suspension member 76.

In use, the end of the cradling member 52 and the suspension member 76 are threaded through the locking member 266, yarned through the bores 288 and 290 respectively, then extending through the rear end 274 and through the bores 287 (FIG. 308A). This step is carried out at a step corresponding with the step illustrated in FIG. 3E. Then, the ends of the cradling member 52 and of the suspension member 76 are manually tensioned (FIG. 8A) to the desired position of the breast.

Upon squeezing trigger 256 (FIG. 8B) the plug-stem 270 is axially displaced in a rear direction, forcing the plug 278 to engage with the locking member 266, clamping therebetween the cradling member 52 and of the suspension member 76, at their tensioned position. Further squeezing of trigger 256 entails rupture of the tear zone 279 giving rise to a clamping unit 300 consisting of the plug 278 and the locking member 266.

Then, pusher 286 is axially pressed in a forward direction as illustrated by arrow 294, entailing the trimming end 282 to shear the cradling member 52 and of the suspension member 76 (FIG. 8C). The tool may now be removed, discharging the clamping unit 300 and leaving it within the breast (not shown).

The tool 250 may be disposable or, according to a different embodiment may be made of a material suitable for reuse (after sterilization), where spare plugs (and their associated plug-stems) are provided.

The system of the present invention generally comprises a cradling member and an anchoring system. The anchoring system secures the cradling member in a desired position and is fixed to a posture tissue in at least one location.

A posture tissue according to the present invention may be a bone (i.e. ribs, fascia or collar bone in the case of breast tissue, pelvic bone in a case of buttocks tissue, humerus bone in case of arm tissue and mandibular bone in case of neck tissue), though it may also be a muscle tissue.

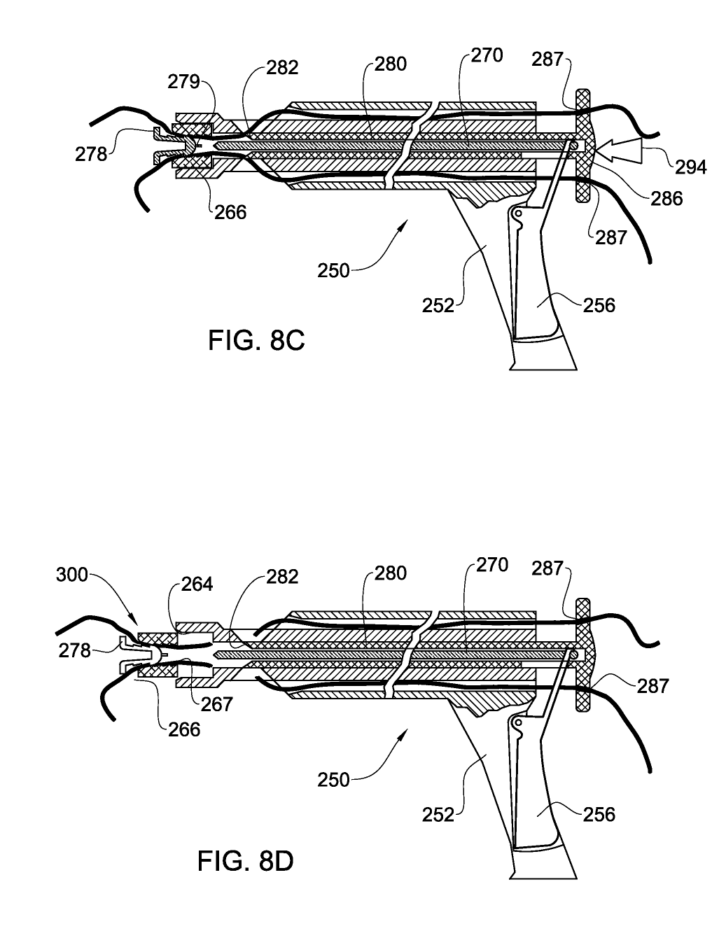

Referring now to FIG. 9A-9B, a soft tissue shaping system 310 according to one example of the present invention comprises a cradling member 312 for cradling a breast tissue and an anchoring system 314 for fixing the cradling member 312 to posture tissue, i.e. ribs, fascia or collar bone.

The anchoring system 314 comprises two anchors 327 (best seen in FIG. 9D) configured to be fixed to a posture tissue and three support members in the form of tabs 317 provided on the cradling member.

The cradling member 312 and the support members 317 may be formed as an integrated homogeneous unit formed from a flexible biocompatible material. According to the exemplified system, the cradling member 312 and the support members 317 are formed seamlessly, for example by molding, in order to eliminate seams, sharp edges, mold flashing and the like. According to another example, the support members 317 may be non-integrated with the cradling member 312 and articulated thereto by other arrangements.

The cradling member 312 may be typically formed to appear rounded, hemispherical or anatomically shaped. In the presently contemplated example, the cradling member 312 is substantially crescent-like shaped and the anchoring system 314 comprises three flat anchoring tabs 317 laterally projecting from a rear longitudinal edge 322 of the cradling member 312 (as best seen in FIG. 9B) which in use is adapted to lie along the infra-mammary fold I (best seen in FIG. 9A). These anchoring tabs 317 secure the cradling member 312 to the individual's posture tissue and substantially prevent its movement during and after the procedure. The anchoring tabs 317 are provided with apertures 318 which may be formed during the manufacturing process or simply by perforation during the process of anchoring the tabs 317 to the posture tissue by appropriate anchors such as self tapping screws, stitches and the like. According to another example, the cradling member may be provided with additional apertures 319 adapted to receive suspending members therethrough (seen in FIG. 9A). The suspending members are in a form of a cord made from a biocompatible material and suspended from an anchor fixed to a posture tissue.

It is desirable that the cradling member 312 and the support members 317 be as thin as possible without compromising its effectiveness to support the soft tissue such as the breast or the buttocks tissues. A cradling member having a thickness ranging from 0.5 millimeters through 3 millimeters is contemplated as being sufficient to support tissue and is believed to function satisfactorily in a majority of cases. The cradling member material may be entirely or partially reinforced with reinforcing ribs or any suitable reinforcing material such as reinforced silicon mesh material. Alternatively, the cradling member may be made of a reinforced silicon mesh material, which may have a varying degree of reinforcement, e.g. the density of the reinforcement may vary across the cradling member.

The cradling member may be made from rigid though pliable material such that it may be deformed to any desired shape and retain its deformed shape for complying with the individual's anatomy and required resultant shape. For example, the cradling member may be made of, or reinforced by, a memory shaped metal, plastic material deformed under heat, a biocompatible material (e.g. in a form of a mesh-like material), silicon sheet, silicon sheet embedded with a mesh-like material, reinforced silicon material, silicon sheet with mesh-like material with non-homogeneous reinforcing qualities i.e., having different mechanical properties (shown in FIG. 9C).

As illustrated in FIG. 9C, the cradling member is a silicon sheet comprising embedded mesh-like material with non-homogeneous reinforcing qualities. The portion of the cradling member designated A has different mechanical properties from the areas designated B. Areas B are adapted to sustain gravitational forces on the cradling member and maintain its shape. The support members according to an example of the invention may also be reinforced to provide a better support.

The soft tissue shaping system 310 may be utilized to support any desired soft tissue. According to one example shown in FIG. 9A the soft tissue is breast tissue 321 and the system 310 is positioned such that the cradling member 312 cradles the breast 321 from below in a manner that the cradling member 312 is aligned and secured along the inframammary fold I.

In the example illustrated in FIG. 9A, the cradling member 312 is substantially crescent shaped and is fitted with two suspension members 345 in the form of cords (e.g. at least partially formed from a tendon-like wire or a mash, made of polyethylene, polyester, polyblend, organic material such as tendons, or synthetic material e.g. silicone, Gortex™, etc.) and corresponding anchors 327 fixed to a posture tissue, above a nipple level. Adjusting the length (shortening/lengthening) of the suspension members 345 entails corresponding lifting or lowering of the breast 321.

The suspension members in accordance with this example and the example illustrated in FIG. 9D are stitched into the end portion 315 of the cradling member 312. In the schematic illustration shown in FIG. 9D, the anchor is a self tapping screw and in order to distribute the load exerted by the soft tissue on the cradling member and the area of connection of suspension member and the cradling member, each anchoring member is fitted with two suspension members (or one suspension member divided into two parts, e.g. by passing it through an eyelet/aperture in the anchor or by coiling it around the anchor). In addition, in order to further distribute the load, the suspension member is sewn into the end portion 315 using several stitches (as also seen in FIGS. 10A and 10B). The stitching is such that if so desired, the length of the suspension members may be manipulated so as to lift the breast to a desired level by pulling on free ends 347 of the suspension members 345. In order to prevent damage to the end portions of the cradling member, these may be reinforced, e.g. as described with reference to FIG. 9C.

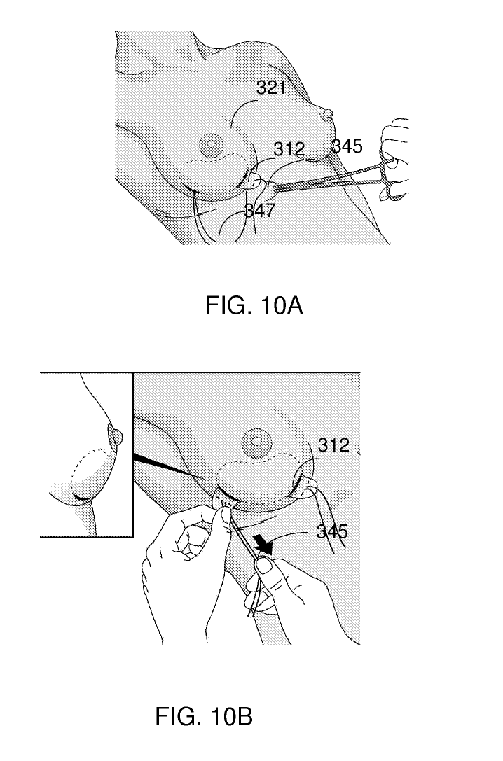

The embodiments of FIGS. 11A and 11B illustrate how the system and the method according to the present invention may be utilized for reshaping buttocks tissue. The system 380 and method is substantially similar to the disclosed hereinabove system and method described with reference to a breast tissue. Owing to the nature and anatomy of the buttocks tissue and its location, it is appreciated that modifications are required such as reinforcement of the cradling member 382 and the anchoring system 384 and the provision of multiple anchoring sites for securing the system to a pelvic bone.

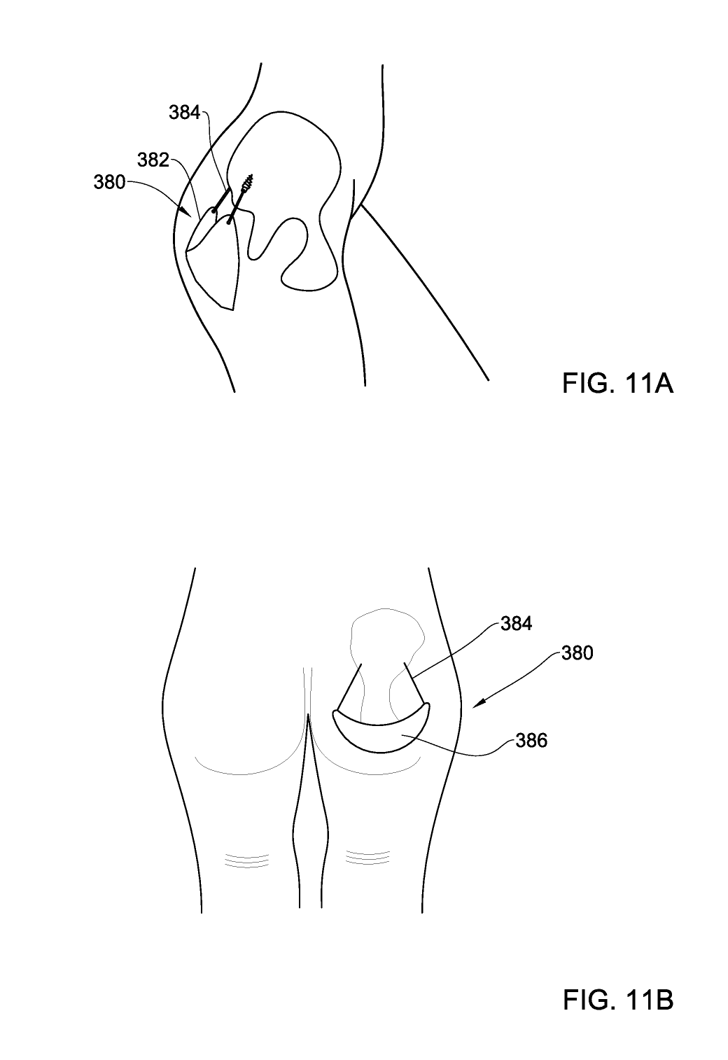

FIG. 12 is a schematic illustration of yet another implementation of the invention showing the system as described hereinabove for reshaping and lifting arm tissue. According to this example, the system, generally designated 400, has a cradling member 410 for cradling loose skin and excess fat tissue and an anchoring system 420 for fixing the cradling member 410 to posture tissue, i.e. a humerus bone.

As shown in FIG. 12 the cradling member 410 is suspended from the posture tissue 430. The anchoring system comprises two suspension members 425 and 426 in the form of cords (e.g. at least partially formed from a tendon like wire or a mesh, made of polyethylene, polyester, polyblend, organic material such as tendons, or synthetic materials such as silicone, etc.) and corresponding anchors 427 and 428 fixed to the posture tissue 430. The cords 425 and 426 may be adjusted in length and position to entail corresponding lifting or lowering of the soft tissue.

As mentioned hereinabove, the cradling member 410 may be formed to undertake a desired anatomical shape and may be made of various materials and designed substantially as described above.

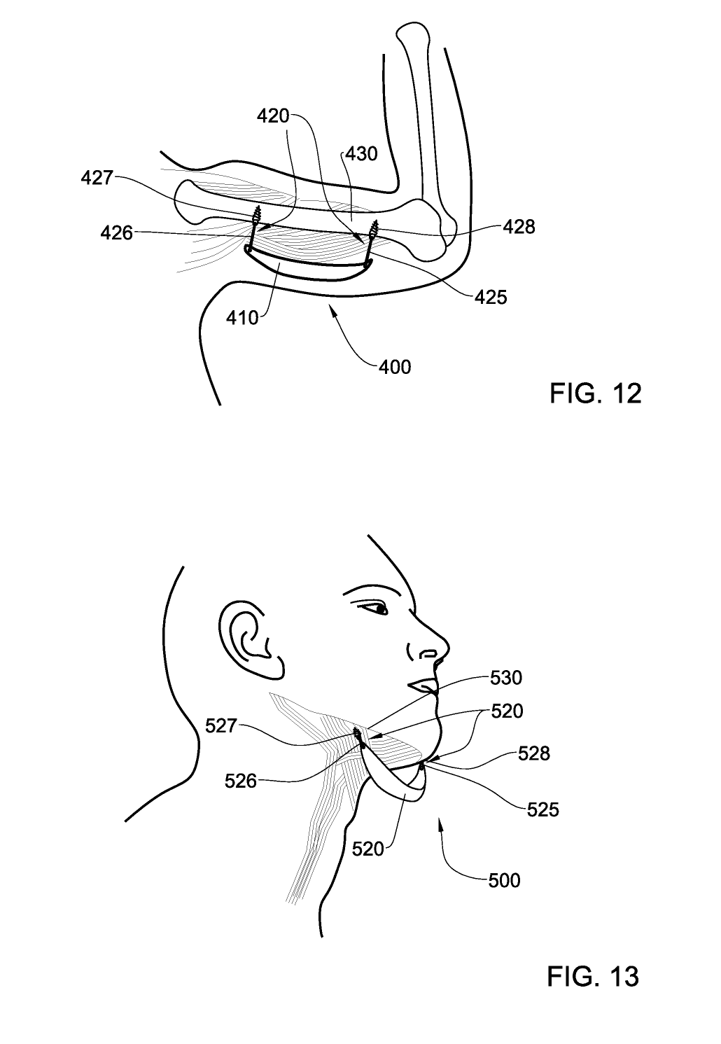

FIG. 13 is a schematic illustration of yet an implementation of the invention showing the system as described hereinabove for reshaping and lifting neck tissue. According to this example, the system, generally designated 500, has a cradling member 510 for cradling loose skin and excess fat tissue and an anchoring system 520 for fixing the cradling member 510 to posture tissue, i.e. mandibular bone.

As shown in FIG. 13, the cradling member 510 is suspended from the posture tissue 530. The anchoring system comprises two suspension members 525 and 526 in the form of cords (e.g. at least partially formed from a tendon like wire or a mesh, made of polyethylene, polyester, polyblend, organic material such as tendons, or synthetic materials such as silicone, etc.) and corresponding anchors 527 and 528 fixed to the posture tissue 530. The cords 525 and 526 may be adjusted in length and position to entail corresponding lifting or lowering of the soft tissue.

As already mentioned hereinabove, the cradling member 510 may formed to undertake a desired anatomical shape and may be made of various materials and designed substantially as described herein.

Whilst some embodiments have been described and illustrated with reference to some drawings, it will be appreciated that many changes may be made therein without departing from the general spirit and scope of the invention, mutatis, mutandis.

1. A system for reshaping and lifting a soft tissue, the system comprising:
a cradling member having a first end and a second end and being configured to cradle the soft tissue;
an anchoring system comprising one or more anchors adapted to be fixed to a posture tissue located above a center of gravity of the soft tissue;
two or more readily adjustable suspension members suspended from the one or more anchors, each readily adjustable suspension member having a free end; and
wherein, at least one of the first end and the second end of the cradling member is integral with and/or movably attached to the free end of a respective suspension member, and the cradling member having a width greater than the width of each of the two or more readily adjustable suspension members, the system being configured to lift, reshape and thereby impart the soft tissue with a desired shape.

2. A system according to claim 1, wherein the anchoring system comprises one or more support members integral with, or articulated to, the cradling member.

3. A system according to claim 2, wherein the support members are in the form of tabs laterally projecting from a longitudinal edge of the cradling member fitted for bearing against posture tissue, soft tissue or fascia.

4. A system according to claim 1, wherein at least a portion of the cradling member is a mesh-like portion.

5. A system according to claim 1, wherein the cradling member is at least partially reinforced.

6. A system according to claim 5, wherein the first and second ends of the cradling member are reinforced.

7. A system in accordance with claim 1, wherein the cradling member is formed from a silicon material embedded with a mesh-like material having reinforcing qualities.

8. A system in accordance with claim 1, wherein the cradling member is homogeneously reinforced.

9. A system in accordance with claim 1, wherein the cradling member is non-homogeneously reinforced.

10. A system in accordance with claim 1, wherein the soft tissue is buttocks tissue.

11. A system in accordance with claim 1, wherein the soft tissue is any one of the buttocks tissue, neck tissue, arm tissue, breast tissue.

12. A system in accordance with claim 1, wherein the one or more suspending members are attached to the cradling member such that the load exerted on a suspending member-cradling member connecting point is spread over a portion of the cradling member.

13. A system for reshaping and lifting a soft tissue, the system comprising:
a cradling member having a first end and a second end;
an anchoring system comprising one or more anchors adapted to be fixed to a posture tissue above a center of gravity of the soft tissue;
two or more readily adjustable suspension members having an anchoring end, and a cradling end, the two or more readily adjustable suspension members being suspended from the one or more anchors at the anchoring end and being movably attached or fixed to the one or more anchors;
the first end of the cradling member being movably attached to and/or integral with the cradling end of one of the two or more readily adjustable suspension members, and a second end movably attached to and/or integral with the cradling end of one of the two or more readily adjustable suspension members,
the cradling member being configured to cradle the soft tissue and the cradling member having a width greater than the width of each of the two or more readily adjustable suspension members;
wherein the system is configured to reshape and lift and thereby impart the soft tissue with a desired shape.

14. A system for reshaping and lifting a soft tissue, the system comprising:
a cradling member having a first end and a second end;
an anchoring system comprising one or more anchors adapted to be fixed to a posture tissue above a center of gravity of the soft tissue;
at least one readily adjustable suspension members having at least one an anchoring point, and at least one a cradling point, the at least one readily adjustable suspension members being suspended from the one or more anchors at the at least one anchoring point and being movably attached to, and/or integral with, the one or more anchors;
the first end of the cradling member being movably attached to, and/or integral with, the at least one cradling point of one of the at least one readily adjustable suspension members, and a second end of the cradling member being movably attached to, and/or integral with, the cradling end of one of the at least one readily adjustable suspension members;
the cradling member being configured to cradle the soft tissue and the cradling member having a width greater than the width of each of the two or more readily adjustable suspension members, wherein the system is configured to lift, reshape and thereby impart the soft tissue with a natural shape.
